(19) United States
(12) Patent Application Publication
(10) Pub. No.: US 2004/0105899 A1
(43) Pub. Date: Jun. 3, 2004

(54) Solvent extraction process

(76) Inventors: Paul Alan Dowdle, Sutton St Helens Merseyside (GB); Stuart Corr, Appleton Cheshire (GB); Helen Harris, Widnes Cheshire (GB)

Correspondence Address: Andrew G Kolomayets, Cook Alex McFarron Manzo Cummings & Mehler, Suite 2850, 200 West Adams Street, Chicago, IL, 60606, US

(21) Appl. No.: 10/415,861

(22) PCT Filed: Nov. 6, 2001
(86) PCT No.: PCT/GB01/04904

(30) Foreign Application Priority Data
Nov. 6, 2000 (GB) 0027047.0
Dec. 7, 2000 (GB) 0029969.3

(51) Int. Cl. A61K 35/78

(52) U.S. Cl. 424/725

(57) ABSTRACT

A process for extracting a compound or composition of matter from a raw material containing that compound or composition as a constituent part is described. The process comprises the steps of (1) contacting the raw material with an extraction solvent comprising a heptafluoropropane so as to extract the compound or composition from the raw material into the solvent, and (2) separating the solvent containing the extracted compound or composition from raw material. The process is particularly adapted for extracting flavours, fragrances and neutraceuticals from materials of plant origin.

The present invention relates to a solvent extraction process in which a raw material containing a particular compound or composition is treated with an extraction solvent so as to remove at least a proportion of that compound or composition from the raw material.

Processes for extracting a desired compound or composition from a raw or bulk material which contains that compound or composition as a constituent part using an extraction solvent are known in the art. In these known processes, the raw material is contacted with the extraction solvent, often under vigorous mixing conditions so as to facilitate the dissolution of the desired compound or composition into the extraction solvent, and the resulting solvent liquor containing the desired compound or composition is then separated from the raw material for subsequent processing, e.g. distillation to remove the extraction solvent. Multiple extractions may suitably be carried out on the same raw material sample so as to maximise the amount of the desired compound or composition which is extracted from that sample. Typical examples of extraction solvents which have been used in the prior art extraction processes include hexane, methyl acetate, ethyl acetate, acetone and methanol.

Although solvent extraction processes are used on a commercial scale, the extraction solvents which are currently used in these processes are not wholly satisfactory. Thus, when solvents such as hexane are used to extract flavoured or aromatic oils, such as are used in the food and cosmetic industries, from plant matter containing those oils, unwanted materials contained in the plant, e.g. high molecular weight waxes, tend to be eluted along with the desired oil. This problem necessitates subjecting the resultant hexane liquor or product concentrate to further processing in which the unwanted components are removed by extraction, e.g. using ethanol. Furthermore, the extraction solvents which are currently in use have fairly high boiling points, and the elevated temperatures which are employed in the distillation process to remove these high boiling solvents from the extracted material can cause problems. For example, the flavoured or aromatic oils contained in certain plants are complex substances containing a large number of individual compounds some of which are relatively lo volatile or relatively thermally unstable. Consequently, high distillation temperatures can tend to result in a loss of product either through co-evaportion of the more volatile compounds with the extraction solvent or thermal degradation of the more thermally unstable compounds.

The use of hydrofluorocarbons such as 1,1,1,2-tetrafluoroethane (R-134a) for extracting products such as flavours and fragrances from materials of natural origin is also known from EP-A-616821.

The present invention provides a new solvent extraction process which can be used to extract a wide variety of compounds or compositions from raw or bulk materials of which they form a constituent part. In one particular embodiment, the present invention provides a solvent extraction process which is capable of extracting the flavoured, functional or aromatic oils or components contained in certain plant or culture materials. A particular characteristic of the present process is that it does make use of an adsorbent.

According to the present invention there is provided a process for extracting a compound or composition of matter from a raw material containing that compound or composition as a constituent part, which process comprises the steps of (1) contacting the raw material with an extraction solvent comprising a heptafluoropropane, e.g. 1,1,1,2,3,3,3-heptafluoropropane (R-227ea), so as to extract the compound or composition from the raw material into the solvent and (2) separating the solvent containing the extracted compound or composition from the raw material.

It will be appreciated that the process of the present invention will not necessarily extract all of the desired compound or composition that is contained in the raw material.

In one particular embodiment, the extraction process of the present invention can be used to extract a natural product from a plant material containing that product.

Accordingly, the present invention provides a process for extracting a natural product from a plant material containing that product as a constituent part, which process comprises the steps of (1) contacting the plant material with an extraction solvent comprising a heptafluoropropane so as to extract the natural product from the plant material into the solvent, and (2) separating the solvent containing the extracted natural product from the plant material.

When used in this specification, the expression “plant material” not only includes materials which are essentially unprocessed and as such are clearly recognisable as being of plant origin, for example bark, leaves, flowers, roots and seeds, but also materials, which although originating from plants, have been subjected to various processes and as such have a form which is somewhat different than the plants from which they originated, for example ground, dried roots or seeds, such as ground cumin and ground ginger, and expressed oils.

In a particularly preferred embodiment, the process of the present invention is used to obtain an extract, such as an essential oil, a concrete or an oleoresin, especially an essential oil, comprising one or more flavour and/or fragrance compounds (hereinafter referred to collectively as organoleptic compounds) from a plant material.

By the term “essential oil” we include oils which contain, inter alia, one or more terpenes and one or more desired organoleptic compounds, such as the oxygen containing terpenoids. Suitable essential oils which may be extracted in accordance with the process of the present invention include citrus peel oils, such as orange, lemon, lime and grapefruit, peppermint, lavandin, rosemary oil and celery seed oil.

Concretes are usually solid, waxy materials produced by solvent extraction of natural products.

Oleoresins are usually viscous, pasty materials produced by solvent extraction of natural products.

The process of the present invention is particularly suitable for extracting flavoured and/or aromatic materials from ginger, vanilla, cloves, star anise and jasmine.

In a further embodiment, the extraction process of the present invention can be used to extract a biologically active compound, such as a pesticide, a neutraceutical or a pharmaceutical, or a precursor to such a biologically active compound from a raw material containing that compound or precursor, such as a plant material, a cell culture or a fermentation broth.

Accordingly, the present invention provides a process for extracting a composition comprising a biologically active compound or a precursor thereof from a raw material containing that composition as a constituent part, which process comprises the steps of (1) contacting the raw material with an extraction solvent comprising a heptafluoropropane so as to extract the composition from the raw material into the solvent, and (2) separating the solvent containing the extracted composition from the raw material.

Suitable pesticides which may be extracted using the extraction process of the present invention include insecticides such as the pyrethroids.

Suitable pharmaceuticals which may be extracted using the extraction process of the present invention include antibiotics, antimicrobials, antifungals and antivirals, for example the penicillins, the alkaloids, paclitaxel, monensin and cytochalasin. Precursors to these compounds may also be extracted using the extraction process of the present invention.

Suitable neutraceuticals that may be extracted include dietary supplements such as antioxidants and vitamins.

The heptafluoropropane which is employed in the process of the present invention may be 1,1,1,2,3,3,3-heptafluoropropane (R-227ea) or 1,1,1,2,2,3,3-heptafluoropropane (R-227ca). Mixtures of the two heptafluoropropanes may also be employed. The preferred heptafluoropropane is 1,1,1,2,3,3,3-heptafluoropropane (R-227ea).

The extraction solvent which is used in the process of the present invention may also comprise a co-solvent in addition to the heptafluoropropane.

Suitable co-solvents will typically have a boiling point of 80° C. or below, for example in the range of from −85 to 80° C. The preferred co-solvents have a boiling point of 60° C. or below, for example in the range of from −85 to 60° C., preferably 20° C. or below, for example in the range of from −70 to 20° C., and more preferably 10° C. or below, for example in the range of from −60 to 10° C. Mixtures of two or more co-solvents may be used if desired.

The co-solvent is also preferably fluorine-free and more particularly halogen-free.

Preferred co-solvents may be selected from the C₂₋₆, particularly the C₂₋₄ hydrocarbon compounds by which we mean compounds containing only carbon and hydrogen atoms. Suitable hydrocarbons may be aliphatic or alicyclic. Preferred hydrocarbons are the alkanes and cycloalkanes, with alkanes such as ethane, n-propane, i-propane, n-butane and i-butane being especially preferred.

Other preferred halogen free co-solvents include the hydrocarbon ethers, by which we mean compounds having the formula R¹—O—R² in which R¹ and R² are independently hydrocarbyl groups containing only carbon and hydrogen atoms, such as C₁₋₆ and preferably C₁₋₃ alkyl groups. Preferred dialkyl ethers include dimethyl ether, methyl ethyl ether and diethyl ether.

Still further suitable co-solvents may be selected from the amides, sulphoxides, alcohols, ketones, carboxylic acids, carboxylic acid derivatives, inorganic acids and nitro compounds.

Preferred amide co-solvents include the N,N′-dialkylamides and alkylamides, especially dimethylformamide and formamide.

Preferred sulphoxide co-solvents include the dialkylsulphoxides, especially dimethylsulphoxide.

Preferred alcohol co-solvents include the aliphatic alcohols, particularly the alkanols. Preferred alkanols are selected from the C₁₋₆, particularly the C₁₋₃ alkanols, with methanol, ethanol, 1-propanol and 2-propanol being especially preferred.

Preferred ketone co-solvents include the aliphatic ketones, particularly the dialkyl ketones. A particularly preferred dialkyl ketone is acetone.

Preferred carboxylic acid co-solvents include formic acid and acetic acid.

Preferred carboxylic acid derivatives for use as co-solvents include the anhydrides, especially acetic anhydride, and the C₁₋₆, particularly the C₁₋₃ alkyl esters of C₁₋₆, particularly C₁₋₃ alkanoic acids, especially ethyl acetate.

Preferred nitro compounds for use as co-solvents include the nitroalkanes and nitroaryl compounds, with nitromethane and nitrobenzene being especially preferred.

The extraction solvent typically comprises from 50.0 to 100% by weight, e.g. from 50.0 to 99.5% by weight, of a heptafluoropropane and from 0 to 50% by weight, e.g. from 0.5 to 50% by weight, of a co-solvent. Preferred extraction solvents comprise from 70.0 to 100.0% by weight, e.g. from 70.0 to 99.0% by weight, of the heptafluoropropane and from 0 to 30% by weight, e.g. from 1 to 30% by weight, of the co-solvent. Particularly preferred extraction solvents comprise from 80.0 to 100.0% by weight, e.g. from 80.0 to 98.0% by weight, of the heptafluoropropane and from 0 to 20.0% by weight, e.g. from 2.0 to 20.0% by weight, of the co-solvent.

If the co-solvent is a flammable material, then the extraction solvent will preferably comprise sufficient of the heptafluoropropane to render it non-flammable overall. Where the extraction solvent is a blend of one or more compounds, the resulting blend may be zeotropic, azeotropic or azeotrope-like.

The extraction solvent which is used in the process of the present invention may be in liquid, gaseous or vaporous form, but is preferably in liquid form. Since both heptafluoropropanes have boiling points below room temperature, maintaining the solvent in liquid form will involve the application of cooling and/or super-atmospheric pressures.

The preferred extraction solvents comprise only low boiling materials so that removal of the solvent from the solvent liquor containing the extract tends to be relatively facile allowing the distillation to be carried out at relatively low temperatures, e.g. room temperature and below. This, in turn, reduces the risk of loosing desired product either through co-evaporation of the more volatile compounds with the extraction solvent or thermal degradation of the more thermally unstable compounds.

The raw material which is subjected to the present extraction process may be a liquid, e.g. a solution, suspension or emulsion, or a solid. If the raw material is a solid, then the efficiency of the extraction process may be significantly improved by reducing the solid to a finely divided form, such as a powder.

The extraction process of the present invention may be conducted at the supercritical temperature of the extraction solvent, in which case elevated temperatures will need to be employed. Preferably, however, the extraction process is conducted at a temperature in the range of from −60 to 150° C., more preferably in the range of from −40 to 60° C. and particularly in the range of from −30 to 40° C.

The extraction process of the present invention may be conducted at atmospheric or super-atmospheric pressures. The precise operating pressure will depend, inter alia, on the extraction solvent which is used, particularly its boiling point, and whether the extraction process is to be conducted with that solvent in liquid or gaseous form. Preferred operating pressures are in the range of from 0.1 to 200 bar, more preferably in the range of from 0.5 to 30 bar and particularly in the range of from 1 to 15 bar.

The contacting of the extraction solvent with the raw material to be processed may be carried out under vigorous mixing conditions so as to facilitate the dissolution of the material to be extracted into the extraction solvent. The vigorous mixing may be achieved by mechanically shaking the extraction vessel containing the raw material/extraction solvent mixture, by stirring that mixture or by the application of ultrasonic excitation.

After the extraction process of the present invention has been completed, the solvent liquor containing the extract can be distilled to remove the extraction solvent from the extract. The resulting extract may then be used as it is or, alternatively, it may be subjected to one or more further processes, for example to purify the extract or to isolate a given compound or compounds contained in the extract.

The extraction process of the present invention may be operated continuously with the same extraction solvent being used repeatedly. A suitable installation for carrying out a continuous extraction process typically comprises an extraction vessel, a distillation unit, a compressor, a condenser and a suitable arrangement of connecting pipe work. The extraction solvent is first charged to the extraction vessel where it is contacted with the raw material to be processed, possibly under vigorous mixing conditions so as to facilitate the dissolution of the compound or composition to be extracted into the extraction solvent. The resulting solvent liquor containing the extract is then separated from the raw material, e.g. by allowing the liquor to drain through a filter arranged at the bottom of the extraction vessel, and passed to the distillation unit where the extraction solvent is removed by evaporation to leave the extract. The vapour generated in the distillation unit is compressed, e.g. using a diaphragm compressor, and is then delivered to a condenser which returns the extraction solvent to liquid form for recharging to the extraction vessel. With a continuous extraction process of this kind, it is possible to maximise the amount of the extract obtained without subjecting the same raw material sample to a succession of individual extractions. Once the raw material sample is exhausted, it is then removed from the extraction vessel and replaced with a fresh raw material sample.

The present invention is now illustrated but not limited by the following examples.

All the examples relate to the extraction of natural products.

General procedures A, B and C relate to the extraction of solid materials. General procedure D relates to the extraction of liquids.

In Examples 1 to 7 and 27 to 40, the extracts that were obtained are of interest primarily as a flavour and/or a fragrance.

In Examples 8 to 26, the extracts that were obtained are of interest primarily as neutraceuticals.

In referring to the yield of the extract, we are referring to the weight of the extract obtained expressed as a percentage of the weight of the original natural product biomass that was subjected to the extraction.

### General Procedure A

Approximately 40 g of the natural product to be extracted was weighed into a cellulose soxhlet extraction thimble. The cellulose thimble containing the natural product was then placed in a glass soxhlet extractor and a pre-weighed receiver flask was attached in position on the extractor. The extractor was then placed in an autoclave, equipped with a cold finger condenser, and the autoclave was sealed and evacuated.

Approximately 330 g of 1,1,1,2,3,3,3-heptafluoropropane (R-227ea) was transferred from a cylinder into the autoclave via a ball valve which was then resealed. The transfer of solvent into the autoclave raised the pressure in the autoclave. The bottom section of the autoclave was then heated to about 50° C. with a hot air gun and cooling fluid was passed through the cold finger condenser to lower the temperature of the cold finger to about −10° C. The extraction was allowed to proceed for a few hours and during this time the temperature and pressure were monitored to ensure that 15 barg was not exceeded. The solvent refluxes within the soxhlet extractor and solvent condensing on the cold finger drops through the contents of the thimble and into the receiver flask.

On completion of the extraction, the solvent was reclaimed by opening the ball valve and condensing the solvent into a cooled Whitey Bomb. When the pressure in the autoclave reached atmospheric, the autoclave was opened and the soxhlet extractor retrieved. The receiver flask containing the extracted material was then detached and re-weighed to determine the yield.

A sample of the extracted material was then analysed by gas chromatography/mass spectrometry (GC/MS) to determine its composition. No correction for individual component response factors was made. The gas chromatogram was a Perlin-Elmer AutoSystem XL coupled to a Perkin Elmer Q-Mass 910 mass detector and a flame ionisation detector. The machine was equipped with a Chrompack CP SIL 5 column of 50 m length. During the analysis, the column temperature was set at 200° C. and held at this temperature for 30 minutes. The injector temperature was set at 300° C. and the flame ionised detector set at 150° C.

A sample of the extracted material was also sent for sensory evaluation. The sample was diluted to 2% w/w in i-propyl alcohol and this diluted material was then added to a suitable quantity of a syrup (specification: 4° Brix; 150 ppm benzoic acid) which had been previously prepared by dissolving 199.91 kg of granular sugar and 0.89 kg of sodium benzoate (preservative) in 1000 litres of water. The resulting composition was then further diluted with water at a ratio of 1 part to 4 parts water.

The prepared sample was then evaluated by an expert taste panel against selected reference materials. The reference materials were based on the naturally occurring constituents of the natural product in question and were classified into groups by the expert panel. The comments of the panel on the organoleptic characters of the extracts were collected.

### General Procedure B

The natural product to be extracted was weighed into a cellulose soxhlet extraction thimble. The cellulose thimble containing the natural product was then placed in a glass soxhlet extractor and a pre-weighed receiver flask was attached in position on the extractor. The extractor was then placed in an autoclave, equipped with a cold finger condenser, and the autoclave was sealed and evacuated.

Approximately 340 g of 1,1,1,2,3,3,3-heptafluoropropane (R-227ea) was transferred from a cylinder into the autoclave via a ball valve which was then resealed. The transfer of solvent into the autoclave raised the pressure in the autoclave. The bottom section of the autoclave was then heated to lo about 50° C. with a heated water jacket and cooling fluid was passed through the cold finger condenser to lower the temperature of the cold finger to about 10° C. The extraction was allowed to proceed for four hours and during this time the temperature and pressure were monitored to ensure that 15 barg was not exceeded. The solvent refluxes within the soxhlet extractor and solvent condensing on the cold finger drops through the contents of the thimble and into the receiver flask.

On completion of the extraction, the solvent was reclaimed by opening the ball valve and condensing the solvent into a cooled Whitey Bomb. When the pressure in the autoclave reached atmospheric, the autoclave was opened and the soxhlet extractor retrieved. The receiver flask containing the extracted material was then detached and re-weighed to determine the yield.

Where indicated, a sample of the extracted material was then analysed by gas chromatography/mass spectrometry (GC/MS) to determine its composition. No correction for individual component response factors was made. The gas chromatogram was a Perkin-Elmer AutoSystem EL coupled to a Perkin Elmer Q-Mass 910 mass detector and a flame ionisation detector. The machine was equipped with a Chrompack CP SIL 5 column of 50 m length. During the analysis, the column temperature was set at 200° C. and held at this temperature for 30 minutes. The injector temperature was set at 300° C. and the flame ionised detector set at 150° C.

### General Procedure C

The natural product to be extracted was weighed into a cellulose soxhlet extraction thimble and ethanol was then added at a loading of 10% by weight on the weight of the natural product biomass. The cellulose thimble containing the natural product biomass and ethanol was then placed in a glass soxhlet extractor and a pre-weighed receiver flask was attached in position on the extractor. The extractor was then placed in an autoclave, equipped with a cold finger condenser, and the autoclave was sealed and evacuated.

Approximately 340 g of 1,1,1,2,3,3,3-heptafluoropropane (R-227ea) was transferred from a cylinder into the autoclave via a ball valve which was then resealed. The transfer of solvent into the autoclave raised the pressure in the autoclave. The bottom section of the autoclave was then heated to about 50° C. with a heated water jacket and cooling fluid was passed through the cold finger condenser to lower the temperature of the cold finger to about 10° C. The extraction was allowed to proceed for four hours and during this time the temperature and pressure were monitored to ensure that 15 barg was not exceeded. The solvent refluxes within the soxhlet extractor and solvent condensing on the cold finger drops through the contents of the thimble and into the receiver flask.

On completion of the extraction, the R-227ea solvent was reclaimed by opening the ball valve and condensing the solvent into a cooled Whitey Bomb. When the pressure in the autoclave reached atmospheric, the autoclave was opened and the soxhlet extractor retrieved. The receiver flask containing the extracted material and ethanol solvent was then detached and the ethanol removed on a rotary evaporator. The receiver flask was then re-weighed to determine the yield.

### General Procedure D

This is a liquid-liquid extraction.

The liquid natural product to be extracted was weighed into a glass liquid-liquid extraction vessel of the type that is conventionally used in the laboratory with heavy solvent phases. A pre-weighed receiver flask was attached in position on the extraction vessel. The extraction vessel was then placed in an autoclave, equipped with a cold finger condenser, and the autoclave was sealed and evacuated.

Approximately 340 g of 1,1,1,2,3,3,3-heptafluoropropane (R-227ea) was transferred from a cylinder into the autoclave via a ball valve which was then resealed. The transfer of solvent into the autoclave raised the pressure in the autoclave. The bottom section of the autoclave was then heated to about 50° C. with a heated water jacket and cooling fluid was passed through the cold finger condenser to lower the temperature of the cold finger to about 10° C. The extraction was allowed to proceed for two or four hours and during this time the temperature and pressure were monitored to ensure that 15 barg was not exceeded. The solvent refluxes within the extraction vessel and solvent condensing on the cold finger drops into the extraction vessel and then into the receiver flask.

On completion of the extraction, the solvent was reclaimed by opening the ball valve and condensing the solvent into a cooled Whitey Bomb. When the pressure in the autoclave reached atmospheric, the autoclave was opened and the liquid-liquid extraction vessel retrieved. The receiver flask containing the extracted material was then detached and re-weighed to determine the yield.

### EXAMPLE 1

In this example, general procedure A described above was used to extract chopped dried ginger. The extraction was continued for 4.5 hours.

A yellow/orange translucent liquid was obtained at a yield of 4.03%. The GC/MS analysis showed that the liquid had the following composition.

| | | |
|---|---|---|
| Pinene | 0.12 | wt. % |
| Camphene | 1.12 | wt. % |
| Phellanderene | 0.21 | wt. % |
| Limonene | 0.02 | wt. % |
| Sabiene | 2.89 | wt. % |
| Eucalyptol | 0.05 | wt. % |
| Cedrene | 14.75 | wt. % |
| Caryophyllene/Farnesene | 19.06 | wt. % |
| Zingibrene | 56.55 | wt. % |
| Bisabolene | 5.23 | wt. % |

The sensory evaluation of the liquid ginger extract was made against the following reference materials.

(i) Earthy notes—Camphene, Bornyl acetate

(ii) Citrus notes—Citral

(iii) Floral notes—Linalool, Geraniol

(iv) Spicy notes—Zingerone, Bisabolene

The extract was classified as follows:

Strong floral/lemon character. High (warm) spice notes. Medium to high heat. Low earthy character. Some phenolic notes.

### EXAMPLE 2

In this example, general procedure A described above was used to extract chopped vanilla pods. The vanilla pods were chopped into approximately 3 mm pieces and the pieces placed in the extraction thimble for extraction in accordance with the general procedure. The extraction was continued for 4.5 hours.

A pale yellow solid was obtained at a yield of 2.83%. The GC/MS analysis showed that the extract had the following composition.

| | | |
|---|---|---|
| 4-hydroxybenzaldehyde | 0.78 | wt. % |
| Vanillin | 99.22 | wt. % |

### EXAMPLE 3

In this example, general procedure A described above was used to extract ground cloves. The extraction was continued for 2 hours.

A pale yellow translucent liquid was obtained at a yield of 9.59%. The GC/MS analysis showed that the liquid had the following composition.

| | | |
|---|---|---|
| Eugenol | 57.59 | wt. % |
| Caryophyllene | 16.89 | wt. % |
| α-Caryophyllene | 0.53 | wt. % |
| Eugenol acetate | 25.0 | wt. % |

The sensory evaluation of the liquid clove extract was made against eugenol, iso-eugenol, eugenyl acetate and clove terpenes.

The extract was classified as follows:

Eugenol, slight phenolic, slight petrol, iso-eugenol, sweet, balsamic, terpenic.

### EXAMPLE 4

In this example, general procedure A described above was used to extract ground star anise. The extraction was continued for 2 hours.

A pale yellow/green oil was obtained at a yield of 5.77%. The GC/MS analysis showed that the oil had the following composition.

| | | |
|---|---|---|
| Limonene | 1.74 | wt. % |
| p-anisaldehyde | 0.39 | wt. % |
| p-allylanisole | 97.87 | wt. % |

The sensory evaluation of the oily star anise extract was made against anethole and anisaldehyde.

The extract was classified as follows:

Clean anethole. Sweet.

### EXAMPLE 5

In this example, general procedure A described above was used to extract jasmine. Jasmine concrete (11.4 g) was melted and loaded onto a particulate vermiculite support (4.5 g). The jasmine coated vermiculite was then charged to the extraction thimble and the extraction conducted in accordance with the general procedure. The extraction was continued for 5.25 hours.

A yellow translucent liquid was obtained at a yield of 41.32%. The GC/MS analysis showed that the liquid had the following composition.

| | | |
|---|---|---|
| Linalool | 24.42 | wt. % |
| Benzyl acetate | 63.29 | wt. % |
| Indole | 1.15 | wt. % |
| Eugenol | 4.5 | wt. % |
| Jasmone | 3.91 | wt. % |
| α-Farnesene | 1.13 | wt. % |
| Unknown | 1.6 | wt. % |

The sensory evaluation of the liquid jasmine extract was made against the following reference materials.

Floral notes

Geraniol (rosey/turkish delight)

Linalool (rosey)

Phenyl ethyl alcohol (aromatic floral/honey-like)

Other

Indole (animal-like)

Cis-Jasmonate (jasmone)

Benzyl acetate (fruity)

The extract was classified as follows:

Strong floral (PEA) with strong jasmone and benzyl alcohol. Slight rosey (linalool) with some fruity character.

### EXAMPLE 6

In this example, general procedure A described above was used to extract ground coffee. The extraction was continued for four hours.

A yellow/orange solid was obtained at a yield of 3.6%.

### EXAMPLE 7

In this example, general procedure A described above was used to extract ground patchouli leaf. The extraction was continued for four hours.

A pale yellow, translucent liquid was obtained at a yield of 11.15%.

### EXAMPLE 8

In this example, general procedure B described above was used to extract 29.9 g of dried, ground rosemary leaves.

A yellow/orange, waxy oil was obtained at a yield of 4.18%.

### EXAMPLE 9

In this example, general procedure B described above was used to extract 50.05 g of green tea leaves.

A green/orange solid was obtained at a yield of 2.5%.

### EXAMPLE 10

In this example, general procedure B described above was used to extract 49.9 g of black tea leaves.

A dark green solid was obtained at a yield of 2.2%.

### EXAMPLE 11

In this example, general procedure B described above was used to extract 60.0 g of ground turmeric.

A pale orange, translucent liquid was obtained at a yield of 4.58%.

### EXAMPLE 12

In this example, general procedure B described above was used to extract 50.95 g of ground, dried soya beans.

A colourless liquid was obtained at a yield of 1.57%.

### EXAMPLE 13

In this example, general procedure B described above was used to extract 50.0 g of powdered valerian root.

A yellow, waxy oil was obtained at a yield of 0.9%.

### EXAMPLE 14

In this example, general procedure B described above was used to extract 50.05 g of dried, powdered St John's Wort flower.

A yellow, waxy material was obtained at a yield of 2.4%.

### EXAMPLE 15

In this example, general procedure B described above was used to extract 40.0 g of dried, powdered Echinacea flower.

White foam like droplets were obtained at a yield of 3.0%.

### EXAMPLE 16

In this example, general procedure B described above was used to extract 35.1 g of dried, powdered ginkgo biloba.

An orange, waxy oil was obtained at a yield of 3.14%.

### EXAMPLE 17

In this example, general procedure B described above was used to extract 69.7 g of dried, powdered Panax ginseng root.

A slightly yellow oily material was obtained at a yield of 1.87%.

### EXAMPLE 18

In this example, general procedure C described above was used to extract 30.05 g of dried, ground rosemary leaves.

A yellow/orange, translucent liquid was obtained at a yield of 1.35%.

### EXAMPLE 19

In this example, general procedure C described above was used to extract 50.55 g of green tea leaves.

A dark green liquid was obtained at a yield of 0.36%.

### EXAMPLE 20

In this example, general procedure C described above was used to extract 49.95 g of black tea leaves.

A dark green liquid was obtained at a yield of 0.23%.

### EXAMPLE 21

In this example, general procedure C described above was used to extract 60.01 g of ground turmeric.

An orange, translucent liquid was obtained at a yield of 3.68%.

### EXAMPLE 22

In this example, general procedure C described above was used to extract 50.35 g of ground, dried soya beans.

A pale yellow, oily emulsion was obtained at a yield of 0.84%.

### EXAMPLE 23

In this example, general procedure C described above was used to extract 49.35 g of powdered valerian root.

A dark green/brown liquid was obtained at a yield of 0.71%.

### EXAMPLE 24

In this example, general procedure C described above was used to extract 49.9 g of dried, powdered St John's Wort flower.

A dark green liquid was obtained at a yield of 2.8%.

### EXAMPLE 25

In this example, general procedure C described above was used to extract 40.1 g of dried, powdered Echinacea flower.

A yellow, translucent liquid was obtained at a yield of 0.96%.

### EXAMPLE 26

In this example, general procedure C described above was used to extract 34.95 g of dried, powdered ginkgo biloba.

A dark green liquid was obtained at a yield of 1.72%.

### EXAMPLE 27

In this example, general procedure B described above was used to extract 1.2 g of rose concrete.

A pink, fragrant oil was obtained at a yield of 8.0%.

A GC/MS analysis was conducted on the oil. The oil contained the following components.

Phenylethyl alcohol

β-Citronellol

Nerol

Geraniol

### EXAMPLE 28

In this example, general procedure B described above was used to extract 1.75 g of oakmoss concrete.

A colourless, fragrant oil was obtained at a yield of 45.7%.

### EXAMPLE 29

In this example, general procedure B described above was used to extract 0.62 g of broom concrete.

A colourless, fragrant oil was obtained.

### EXAMPLE 30

In this example, general procedure B described above was used to extract 43.9 g of ground West African cocoa beans.

A white, waxy solid having an intense cocoa fragrance was obtained at a yield of 2.0%.

### EXAMPLE 31

In this example, general procedure B described above was used to extract 44.1 g of ground Indian black pepper.

A pale yellow oil with white solids was obtained at a yield of 5.0%.

A GC/MS analysis was conducted on the composition. The composition contained the following components.

α-Pinene

Sabinene

β-Pinene

3-Carene

Limonene

β-Phellandrene

Copaene

Caryophyllene

### EXAMPLE 32

In this example, general procedure B described above was used to extract 43.5 g of ground pink pepper.

A pale yellow oil with white solids was obtained at a yield of 5.5%.

A GC/MS analysis was conducted on the composition. The composition contained the following components.

α-Pinene

Sabinene

β-Myrcene

β-Pinene

α-Phellandrene

3-Carene

Limonene

Linalyl acetate

β-Phellandrene

Copaene

Caryophyllene

Germacrene

### EXAMPLE 33

In this example, general procedure B described above was used to extract 31.65 g of ground Szechuan pepper.

A pale yellow oil with crystalline solids was obtained at a yield of 4.6%.

A GC/MS analysis was conducted on the composition. The composition contained the following components.

α-Pinene

Sabinene

β-trans Ocimene

p-Cymene

Limonene

β-Phellandrene

cis-Thujan-4-ol

Linalool

Caryomenthone

1-(4-hydroxy-3,5-dimethoxyphenyl)-ethanone

### EXAMPLE 34

In this example, general procedure B described above was used to extract 12.2 g of crushed, dried mint leaves.

A pale green oil was obtained at a yield of 7.8%.

A GC/MS analysis was conducted on the oil. The oil contained the following components.

Eucalyptol

trans-Thujan-4-ol

p-Menthone

Isomenthone

Neomenthol

Menthol

Pulegone

3-Carvomenthone

Menthyl acetate

β-Farnesene

Caryophyllene

### EXAMPLE 35

In this example, general procedure B described above was used to extract 26.7 g of ground magnolia bark.

A yellow oil was obtained at a yield of 5.6%

A GC/MS analysis was conducted on the oil. The oil contained the following components.

Caryophyllene

β-Selinene

Caryophyllene oxide

Eudesmol

β-Eudesmol

### EXAMPLE 36

In this example, general procedure C described above was used to extract 1.45 g of rose concrete.

A pink, fragrant oil was obtained at a yield of 3.6%.

### EXAMPLE 37

In this example, general procedure C described above was used to extract 29.8 g of ground magnolia bark.

A yellow oil was obtained at a yield of 2.3%.

### EXAMPLE 38

In this example, general procedure D described above was used to extract 33.9 g of Californian white grapefruit oil. The extraction was continued for 2 hours.

A pale yellow oil was obtained at a yield of 43.7%. The oil was paler in colour and had a stronger fruit aroma than the original oil.

### EXAMPLE 39

In this example, general procedure D described above was used to extract 42.8 g of Sicilian CP lemon oil. The extraction was continued for 2 hours.

A pale yellow oil was obtained at a yield of 84.6%. The oil was paler in colour and had a stronger fruit aroma than the original oil.

### EXAMPLE 40

In this example, general procedure D described above was used to extract 40.9 g of Mexican lime oil. The extraction was continued for 4 hours.

An oil having similar properties to the original oil was obtained at a yield of 94%.

1. A process for extracting a compound or composition of matter from a raw material containing that compound or composition as a constituent part, which process comprises the steps of (1) contacting the raw material with an extraction solvent comprising a heptafluoropropane so as to extract the compound or composition from the raw material into the solvent, and (2) separating the solvent containing the extracted compound or composition from the raw material.

2. A process as claimed in claim 1, wherein the raw material is of plant origin and the composition to be extracted is a flavour or a fragrance.

3. A process as claimed in claim 2, wherein the flavour or fragrance is comprised in an oil, a concrete or an oleoresin.

4. A process as claimed in claim 1, wherein the compound or composition to be extracted from the raw material comprises a biologically active compound or a precursor thereof.

5. A process as claimed in claim 4, wherein the biologically active compound is a pesticide or a precursor thereof.

6. A process as claimed in claim 4, wherein the biologically active compound is a pharmaceutically active substance or a precursor thereof.

7. A process as claimed in claim 4, wherein the biologically active compound is a neutraceutical or a precursor thereof.

8. A process as claimed in any one of the preceding claims, wherein the extraction solvent comprises 1,1,1,2,3,3,3-heptafluoropropane (R-227ea).

9. A process as claimed in any one of the preceding claims, wherein the extraction solvent comprises a co-solvent in addition to the heptafluoropropane.

10. A process as claimed in claim 9, wherein the co-solvent comprises at least one C₂₋₆ hydrocarbon.

11. A process as claimed in claim 10, wherein the C₂₋₆ hydrocarbon is selected from the alkanes and cycloalkanes.

12. A process as claimed in claim 11, wherein the co-solvent comprises at least one compound selected from ethane, n-propane, i-propane, n-butane and i-butane.

13. A process as claimed in claim 12, wherein the co-solvent comprises n-butane.

14. A process as claimed in claim 9, wherein the co-solvent comprises at least one hydrocarbon ether having the formula R¹—O—R² in which R¹ and R² are independently C₁₋₆ alkyl groups.

15. A process as claimed in claim 14, wherein the co-solvent comprises at least one ether selected from dimethyl ether, methyl ethyl ether and diethyl ether.

16. A process as claimed in claim 15, wherein the co-solvent comprises dimethyl ether.

17. A process as claimed in claim 9, wherein the co-solvent comprises at least one compound selected from the C₁₋₆ alkanols and the C₁₋₆ alkyl esters of C₁₋₆ alkanoic acids.

18. A process as claimed in claim 17, wherein the co-solvent is selected from ethanol and ethyl acetate.

19. A process as claimed in any one of the preceding claims, wherein the extraction solvent is in liquid form.

20. A process as claimed in any one of the preceding claims which is conducted at a temperature in the range of from −60 to 150° C.
